(19) United States
(12) Patent Application Publication
(10) Pub. No.: US 2021/0290982 A1
(43) Pub. Date: Sep. 23, 2021

(54) DETERMINING WATER EQUIVALENT PATH LENGTH

(71) Applicant: Oxford University Innovation Limited, Oxford (Oxfordshire) OX2 0JB (GB)

(72) Inventors: Frank VAN DEN HEUVEL, Oxford (Oxfordshire) (GB); Francesca FIORINI, Oxford (Oxfordshire) (GB)

(21) Appl. No.: 17/263,975

(22) PCT Filed: Jul. 29, 2019
(86) PCT No.: PCT/GB2019/052112

§ 371 Date: Jan. 28, 2021

(30) Foreign Application Priority Data
Jul. 30, 2018 (GB) 1812368.7

(51) Int. Cl. A61N 5/10; G01T 1/29; G06N 3/02

(52) U.S. Cl. CPC A61N 5/1075; A61N 5/103; A61N 2005/1087; G06N 3/02; G01T 1/2914

(57) ABSTRACT

A measurement apparatus for determining a water equivalent path length (WEPL) through an object (100), the measurement apparatus comprising a proton beam source (1) arranged to produce, in use, a beam (2) of protons having a beam shape; a proton detector (3), the proton detector (3) defining a proton detection plane, the proton detector (3) being arranged to measure a spatial profile of protons incident the proton detection plane; and energy deposited inside the detector by protons incident on the proton detection plane the proton detector (3) further arranged to provide a signal indicative of the measured energy with the spatial profile; and a processor (4) coupled to the proton detector (3) so as to process the signal; in which the proton beam source (1) and the proton detector (3) define between them a space for the object (100), and in which the processor (4) is arranged to process the signal so as to fit the spatial profile and deposited energy measured after the proton beam (2) has passed through the object (100) to a distribution having parameters, and from the parameters estimate a water equivalent path length of the object (100).

This invention relates to a measurement apparatus for and a method of determining measuring water equivalent path length.

Proton therapy has the potential of being a paradigm shifting treatment modality for cancer. This is due to a physical property of heavier charged particles, which can come to a complete stop in a medium depositing the majority of its energy at the end of its track (Bragg peak). For a given energy of the particle and given medium the range of such a particle is well known to within a few millimetres. This knowledge can be harnessed to produce dose distributions that spare normal tissue better than classical photon-based treatments.

This clear advantage, which is touted in the media, is also the Achilles heel of proton therapy. The sharp distal edge of the Bragg peak implies a criticality which is not present when using photon based treatments. In addition the range depends linearly on the density of the medium, and in a lesser way on the atomic content of the medium (stopping power).

In a realistic treatment the content of the medium is not well known and difficult to measure accurately. In addition it can change drastically due to internal movement within the patient. This uncertainty is increased in environments with complex anatomical structures such as breast, bone, and lung. To cope with the impact of such complexity on the quantity of range, the concept of water equivalent path length (WEPL) was introduced. This is the amount of water needed to have the same reduction of range as the combination of all of the structures passed along the way. This well-known concept can be used for single protons but also for multi-proton pencil beams.

Measuring the WEPL in a patient provides us with valuable information about how the treatment is progressing. Ideally, this measurement should be repeated just before or during the treatment session as it would provide information on the accuracy and safety of the delivered treatment. Indeed, by comparing the WEPL data in an idealized situation (i.e. in a treatment plan), large changes in anatomy which would change the treatment's efficacy drastically can be observed before starting the treatment and possible alternatives or changes to the treatment can be proposed (adaptive treatment). Finally, other groups have proposed to use WEPL measurements to perform reconstructions of internal information using a computer tomography (CT) approach and generate a so-called proton CT. However, the sources and sensors required to generate a CT scan using protons are complex and expensive.

According to a first aspect of the invention, we provide a measurement apparatus for determining a water equivalent path length (WEPL) through an object, the measurement apparatus comprising:
a proton beam source arranged to produce, in use, a beam of protons having a beam shape;
a proton detector, the proton detector defining a proton detection plane, the proton detector being arranged to measure:
   a spatial profile of protons incident on the proton detection plane; and
   energy deposited inside the detector by protons incident on the proton detection plane
the proton detector further arranged to provide a signal indicative of the measured energy with the spatial profile; and
a processor coupled to the proton detector so as to process the signal;

in which the proton beam source and the proton detector define between them a space for the object, and in which the processor is arranged to process the signal so as to fit the spatial profile and deposited energy measured after the proton beam has passed through the object to a distribution having parameters, and from the parameters estimate a water equivalent path length of the object.

As such, we have appreciated that, by using a parameterised distribution, it is unnecessary to use the complex equipment required for (proton) computed tomography in order to determine the WEPL.

In one embodiment, the distribution is a stable distribution. We have found that they are particularly appropriate for the parameterisation of a beam of protons to determine WEPL.

The stable distribution may be defined by its characteristic function, which may be given by:

φ(t; α, β, γ, δ)=exp[itδ−|γt|^{α}(1−iβ sgn(t)ϕ)]

with ϕ(t)=tan(πα/2) except for α=1, where

$$ϕ = - \frac{2}{π} log ( t )$$

where the parameters comprise:
a first parameter, α,
a second parameter, β, and
a third parameter, γ;

and δ represents the position of the beam on the proton detector and sgn(t) is the sign function.

As such, the first parameter may have a value between 0 and 2 inclusive and may be indicative of the shape of the beam as incident on the proton detector. The second parameter may have a value between −1 and 1 inclusive and may be a measure of the symmetry of the beam as incident on the proton detector. The third parameter may have a value greater than or equal to 0 and less than positive infinity, and may represent the broadness of the distribution.

The processor may be arranged to determine an integrated proton dose deposited in the detector by protons incident on the proton detector for the beam, and typically to use the integrated proton dose to determine the WEPL.

The processor may be arranged to determine the WEPL by using the first, second and third parameters and the integrated proton dose as the inputs to a trained neural network. For example, this may be a regressor neural network, which may have an input layer having, in order:
an input layer having a node for each input to the neural network,
at least one hidden layer each having a plurality of nodes;
and an output layer comprising an output node at which the WEPL is output;

in which the nodes of each of the input layer and each hidden layer are each connected by connections to the nodes in the following layer. A weighting may be associated with each connection; the training of the neural network may set those weights.

The proton beam source may be a therapeutic proton beam source; as such, it may be possible to provide a therapeutic proton beam and determine the WEPL without needing a separate imaging radiation source.

The apparatus may further include a second proton detector, provided between the proton beam source and the object. The second proton detector may be arranged to measure:
a spatial profile of protons incident on a proton detection plane of the second proton detector; and
energy deposited inside the second proton detector by protons incident on the second proton detection plane.

The second proton detector may be further arranged to provide a signal indicative of the measured energy with the spatial profile. The processor may be further arranged to: fit the spatial profile and deposited energy measured by the second proton detector before the proton beam has passed through the object to the distribution, and based on a difference between parameters determined before the proton beam passes through the object, and after the proton beam has passes through the object, estimate a water equivalent path length of the object.

The use of the differences between the parameters allows for a more stable and accurate system, as variations or drifts in the source are removed from the calculation.

The proton beam source may be arranged so that the beam shape of the beam as it exits the proton beam source is less than 5 cm wide; the proton beam may therefore be a pencil beam. The proton beam source will typically be arranged so that the proton beam has sufficient energy to pass through the object; that is, the range is greater than the geometric distance the proton beam travels through the object.

The apparatus may comprise a housing of at least one of the proton beam source and the proton detector. As such, the processor may be housed within the housing also. Alternatively, the processor may be separately housed, typically in a separate computer, such as a desktop or laptop computer, or in a server remote from the proton beam source and the proton detector. As such, the analysis can be provided locally or remotely.

The object may be a person or animal—typically a patient—or part thereof.

According to a second aspect of the invention, we provide a method of determining a water equivalent path length (WEPL) through an object, method comprising:
passing a beam of protons having a beam shape through the object; and
detecting, with a proton detector defining a proton detection plane:
   a spatial profile of protons incident on the proton detection plane; and
   energy deposited inside the proton detector by protons incident on the proton detection plane;
in which the proton beam is incident on the proton detection plane after it has passed through the object;
the method further comprising fitting the detected spatial profile and deposited energy to a distribution having parameters and from the parameters estimating a water equivalent path length of the object.

As such, we have appreciated that, by using a parameterised distribution, it is unnecessary to use the complex equipment required for (proton) computed tomography in order to determine the WEPL.

In one embodiment, the distribution is a stable distribution. We have found that they are particularly appropriate for the parameterisation of a beam of protons to determine WEPL.

The stable distribution may be defined by its characteristic function, which may be given by:

φ(t; α, β,γ, δ)=exp[itδ−|γt|^{α}(1−iβ sgn(t)ϕ)]

where the parameters comprise:
a first parameter, α,
a second parameter, β, and
a third parameter, γ;

and δ represents the position of the beam on the proton detector and sgn(t) is the sign function.

As such, the first parameter may have a value between 0 and 2 inclusive and may be indicative of the shape of the beam as incident on the proton detector. The second parameter may have a value between −1 and 1 inclusive and may be measure of the symmetry of the beam as incident on the proton detector. The third parameter may have a value greater than or equal to 0 and less than positive infinity and may represent the broadness of the distribution.

The method may comprise determining an integrated proton dose deposited in the detector by protons incident on the proton detection plane for the beam, and typically using the integrated proton dose to determine the WEPL.

The method may comprise determining the WEPL by using the first, second and third parameters and the integrated proton dose as the inputs to a trained neural network. For example, this may be a regressor neural network, which may have an input layer having, in order:
an input layer having a node for each input to the neural network,
at least one hidden layer each having a plurality of nodes;
and an output layer comprising an output node at which the WEPL is output;

in which the nodes of each of the input layer and each hidden layer are each connected by connections to the nodes in the following layer. A weighting may be associated with each connection; the training of the neural network may set those weights.

The method may comprise training the neural network. The step of training the neural network may comprise training the neural network by providing the first, second and third parameters and the integrated proton dose and the WEPL for sample objects and typically adjusting the weights. The sample objects may have known WEPLs, or the method may comprise measuring the WEPL of the sample objects, typically with alternative techniques such as proton computed tomography. Alternatively, or additionally, at least some of the sample objects may be virtual sample objects, were the WEPL is predicted using a WEPL prediction algorithm. An example of such an algorithm that can be used is the FLUKA Monte Carlo simulation.

The method may include, prior to passing the beam of protons through the object, detecting:
a spatial profile of protons incident on a second proton detection plane; and
energy deposited inside a second proton detector by protons incident on the second proton detection plane.

The method further may further comprise fitting the detected spatial profile and deposited energy to the distribution and from the difference between the parameters detected before and after the beam passes through the object estimating a water equivalent path length of the object.

The use of the differences between the parameters allows for a more stable and accurate system, as variations or drifts in the source are removed from the calculation.

The proton beam may be created by a therapeutic proton beam source; as such, it may be possible to provide a therapeutic proton beam and determine the WEPL without needing a separate imaging radiation source or reconstructing the path followed by the beam inside the object

The beam shape of the beam before it enters the object may be less than 5 cm wide; the proton beam may therefore be a pencil beam. The proton beam will typically have sufficient energy to pass through the object; that is, the range is greater than the distance the proton beam travels through the object.

The object may be a person or animal—typically a patient—or part thereof.

There now follows, by way of example only, description of an embodiment of the present invention, described with reference to the accompanying drawings, in which:

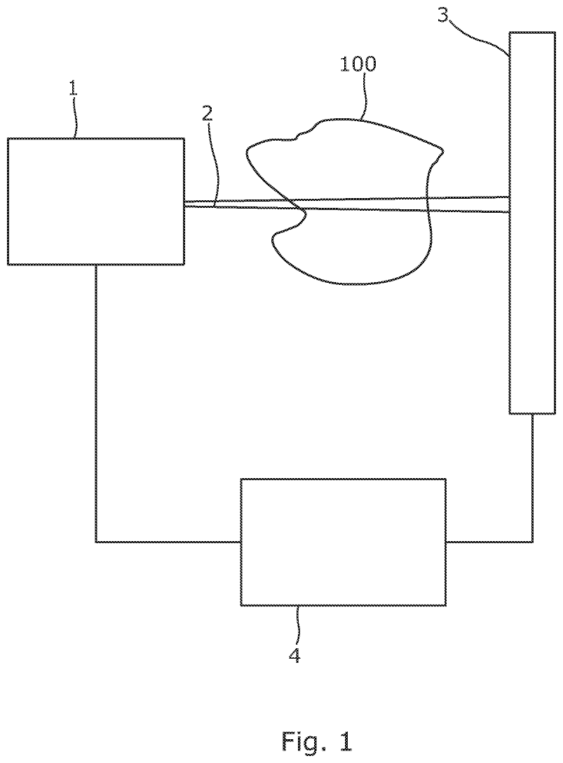

FIG. 1 shows schematically a measurement apparatus in accordance with an embodiment of the invention;

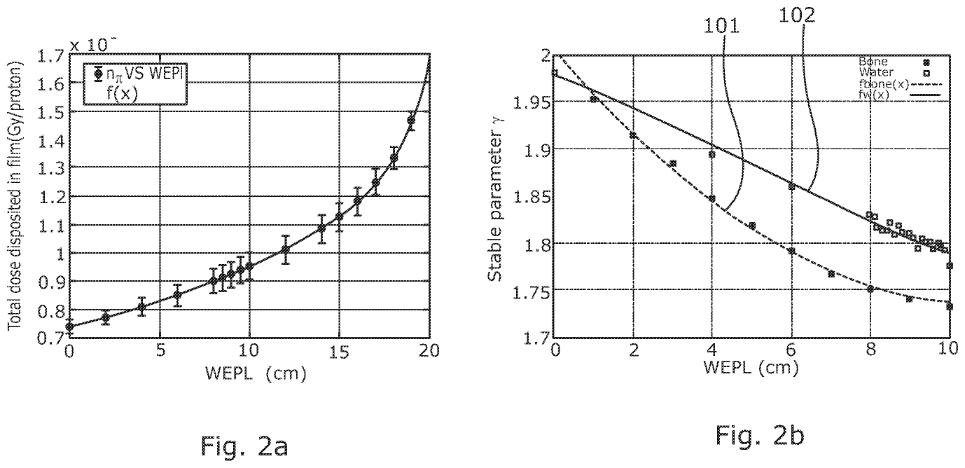

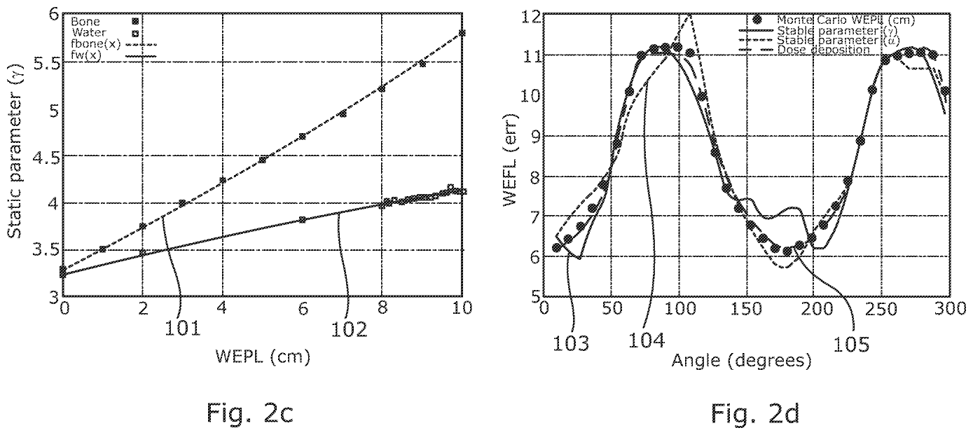

FIGS. 2a to 2d shows graphs of the water equivalent path length (WEPL) with different parameters of the proton beam of the apparatus of FIG. 1;

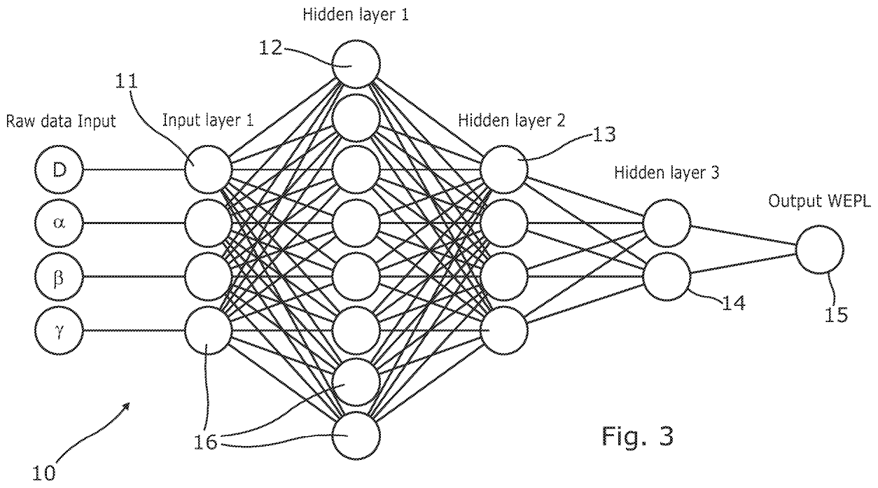

FIG. 3 shows a neural network implemented by the apparatus of FIG. 1;

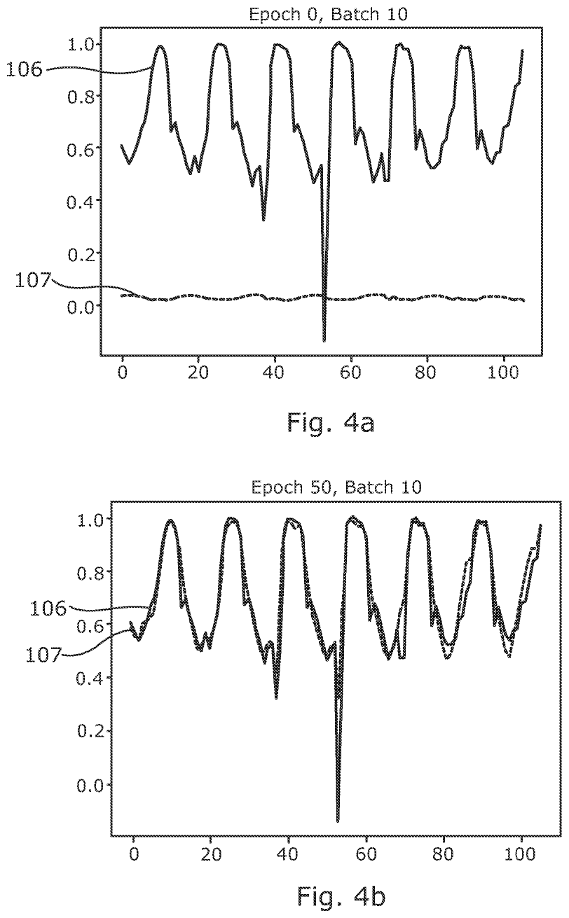

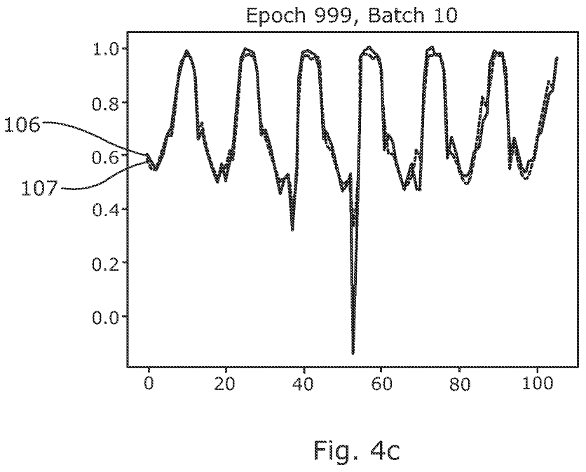

FIGS. 4a to 4c show graphs showing the neural network of FIG. 3 converging on the ideal WEPL;

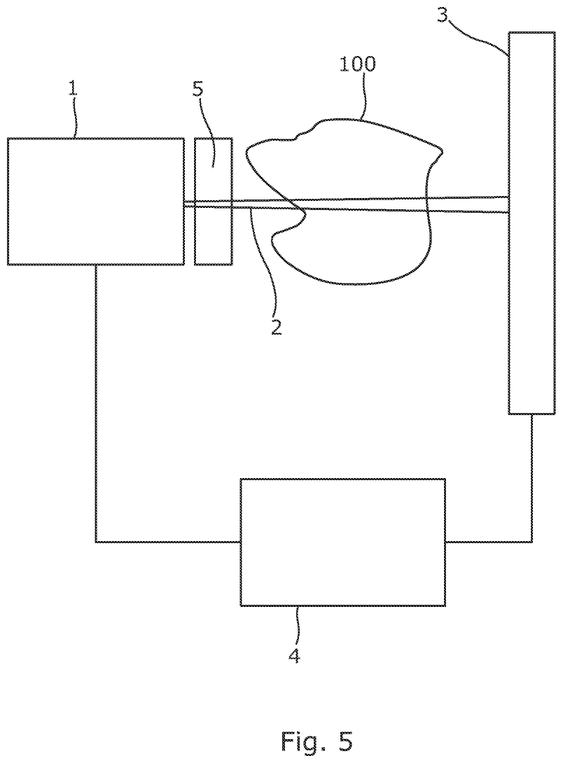

FIG. 5 shows schematically a measurement apparatus in accordance with an alternative embodiment of the invention;

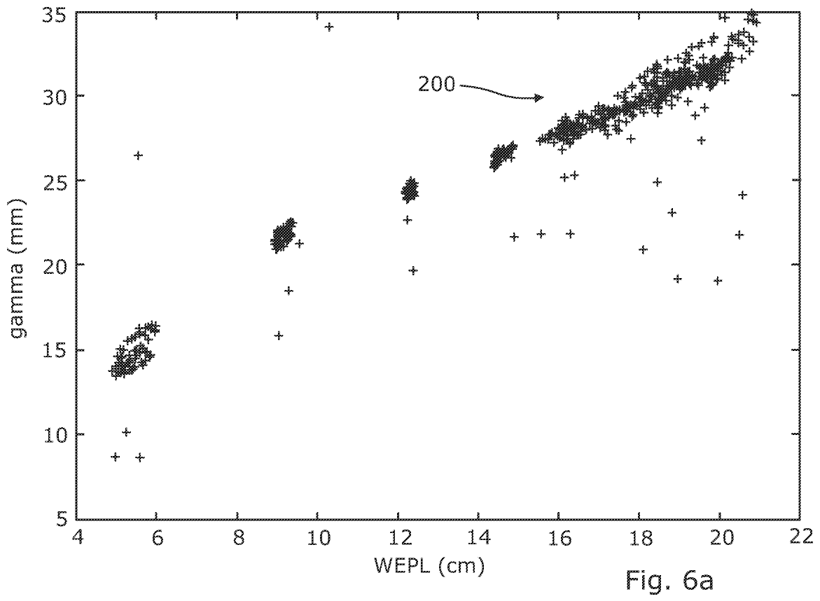

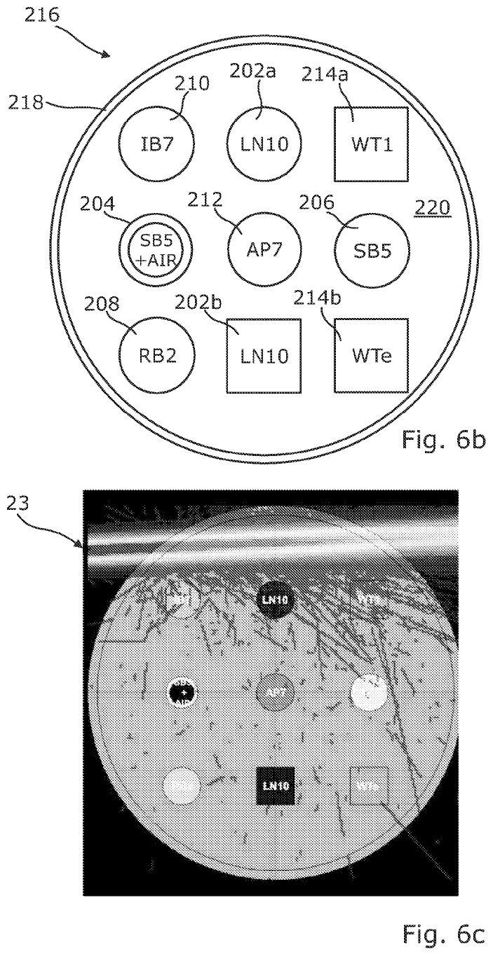

FIG. 6a shows example results for a test phantom, showing an experimentally derived beam parameter as a function of a simulated WEPL;

FIG. 6b schematically illustrates the arrangement of the phantom used for obtaining the results of FIG. 6a; and

FIG. 6c illustrates a representation of the simulation of the experimental results, used to obtain the results of FIG. 6a.

FIG. 1 shows a schematic view of a measurement apparatus in accordance with an embodiment of the invention. It can be used to determine the water equivalent path length (WEPL) of a proton beam as it passes through an object 100, such as a patient.

The apparatus comprises a proton beam source 1, such as a cyclotron or synchrotron accelerated proton beam from a gantry nozzle. For example the proton beam source 1 may be an IBA PROTEUS® PLUS source.

The proton beam source 1 generates a pencil beam of protons 2. This passes through the object 100 and impinges upon a planar proton detector 3, such as a radiochromic film, scintillation plate, or silicon based detector. For example, the proton detector 3 may be a MediPIX detector.

The proton detector 3 determines the spatial profile of protons incident on the plane of the proton detector 3 (ie. the geometric distribution of protons over the plane).

Furthermore, although the proton detector 3 is planar, the proton beam 2 will pass along a transmission path through the thickness of the detector 3. As it passes along this path, the proton beam 2 will transfer energy to the detector 3. The detector 3 measures this energy, also referred to as the dose.

The beam 2 emitted by the proton source 1 has a spatial profile that can be approximated to a stable distribution. As the beam 2 passes through the object 100, the spatial profile of the beam 2 changes. The profile measured at the detector 3 may be approximated to another stable distribution having parameters different from those of the distribution before the object 100. Different objects 100 will cause different changes to the spatial profile. Furthermore, the dose deposited at the detector 3 will vary for different objects 100. These variations can be used to determine the WEPL.

The apparatus comprises a processor 4 which is arranged to process the output of the proton detector 3 in order to determine the WEPL. In order to do this, it fits the profile of the proton beam 2 measured at the detector 3 to a stable distribution. The stable distribution is described by a characteristic function, which is the Fourier transform of the probability density of said stable distribution, and is given by:

φ(t; α, β, γ, δ)=exp[itδ−|γt|^{α}(1−iβ sgn(t)ϕ)]

with ϕ(t)=tan(πα/2) except for α=1, where

$$ϕ = - \frac{2}{π} log ( t ) ,$$

where t is (the frequency in Fourier space), α is between 0 and 2 inclusive and determines the shape of the distribution, β is between −1 and 1 inclusive and is a measure of symmetry, γ is a non-negative real number representing the broadness of the distribution, governed by the beam divergence coupled with the primary scattering process, and δ is indicative of the position of the beam.

The processor finds values for α, β, γ and δ which best fit the observed beam. For example, the best-fit may be found by a maximum likelihood estimation based on pre-computed spline approximations, such as disclosed in Barndorff, Nielsen O E, Mikosch T, Resnick S I, eds. Levy Processes: Theory and Applications. Boston, Mass.: Birkhauser Boston; 2001; 379-40. However, any suitable method may be used to determine the best-fit.

The processor also determines the total dose Dₜₒₜ deposited by the beam 2 inside the proton detector 3, as an integrated proton dose.

We have found that the parameters α, β and γ together with Dₜₒₜ can be used to determine the WEPL of the beam 2 passing through the object 100 as described below. As can be seen in FIG. 2a to 2d, these parameters can be used individually to estimate WEPL. FIGS. 2a to 2c represent the results of a FLUKA Monte Carlo simulation of a proton beam, passing through water test objects (FIG. 2a to 2c) and bone test objects (FIGS. 2b to 2c), also referred to as phantoms. By varying the thickness of the phantoms, it is possible to determine the relationship between α, β, γ and Dₜₒₜ and the WEPL of the crossed materials.

A test study has been carried out using a lamb neck which has been previously scanned with a conventional CT, with the beam being rotated around the isocentre placed inside the object to be able to scan an entire 360 degrees slice of the neck. FIG. 2d illustrates the results of the test study. In FIG. 2d, dots indicate the simulated data points for WEPL, as calculated from α, β, γ and Dₜₒₜ, against the beam angle around the neck. The best fit lines through those points are also shown.

In FIG. 2a, the integrated dose Dₜₒₜ is shown against WEPL, and it can be seen that Dₜₒₜ is sensitive to WEPL particularly at high WEPLs.

In FIG. 2b, the parameter a is shown against the WEPL, for bone (trace 101) and water (trace 102) test objects. This parameter shows better sensitivity for low WEPLs (note the x-axis scale differs to that of FIG. 2a).

In FIG. 2c, the parameter γ is shown against the WEPL in the same manner as in FIG. 2b. Again, this parameter shows better sensitivity for low WEPLs.

FIG. 2d shows WEPLs of the lamb neck slice as estimated by the three parameters mentioned above (α, γ and Dₜₒₜ) against the angle of rotation of the beam around the isocentre in the neck. The dots indicate the simulated WEPL (the ideal value) and the three traces show WEPLs estimated by the parameters α (trace 103), γ (trace 104) and Dₜₒₜ (trace 105). These are all a reasonable fit to the ideal values, and can be estimated by second or third order polynomials of the relevant parameter.

However, given that the parameters give better results at different ranges of WEPL, we propose using a neural network to determine the WEPL, given that it appears that combinations of the four parameters should be able to determine the WEPL over a wide range but with combinations that depend on the WEPL in question. In artificial intelligence the notion of neural networks has recently been made possible due to advances in computer speed and increased storage capacity. In most applications a neural network is used to classify the input, for example the recognition of written characters. In this case we are not interested in a selection problem, but want a quantitative estimate of the value. Such networks are denoted as regressor networks. The architecture of the network is optimized depending on the number of input variables and the complexity of the problem.

FIG. 3 shows an example neural network, which in an exemplary embodiment we have implemented in the Tensorflow library, and which would be implemented as software or hardware in the processor 4. As is common in neural networks, the neural network 10 comprises a number of layers 11, 12, 13, 14, 15 each comprising a number of nodes 16. In this example, an input layer 11 has four nodes 16, one for each of the inputs α, β, γ and Dₜₒₜ, a first hidden layer 12 has 8 nodes 16, a second hidden layer 13 has 4 nodes 16, a third hidden layer 14 has two nodes 2 and an output layer 15 has one node 16 for the output WEPL.

The nodes of one layer are connected to all of the nodes of the following layer, and pass on the values input to them summed with varying weights, with biases being added or subtracted by each node. By changing these weights and biases through a process of training, the neural network 10 can be used to determine the WEPL. Training will take the place of the presentation of the parameters α, β, γ and Dₜₒₜ for a series of test objects and the iterative changing of the weights and biases so that the WEPL predicted by the neural network converges with the ideal WEPL.

This can be seen in FIGS. 4a to 4c of the accompanying drawings, where the ideal WEPL is shown at trace 106 and the WEPL output from the neural network is shown at trace 107 both against an angle through the test object. The weights and biases are initially set (in FIG. 4a) as uniform; it can be seen that after 50 iterations (FIG. 4b) the traces start to converge, whereas after 999 iterations (FIG. 4c) convergence is good.

As such, it can be seen that water equivalent path length map of an object 100 such as a patient can be inferred by analysing the characteristics α, β, γ and Dₜₒₜ of the exiting beam 2, without reconstructing the path the beam followed inside the object 100.

FIG. 6a illustrates an example of experimental data 200 measured on a test phantom 216 to demonstrate the calibration of the system. FIG. 6b illustrates the arrangement of the test phantom 216 used.

The test phantom 216 was a cylinder of 20 cm diameter and 12 cm height. The phantom 216 had a cylindrical outer wall 218 and a bottom wall and top wall (not shown) defining a volume 220. The outer wall 218, and top and bottom walls were of Perspex. Within the volume, nine different sample inserts made of different materials were provided, located in indentations in the bottom wall. The volume 220 was then filled with water.

The inserts were cylindrical and parallelepiped in shape, and arranged in a three-by-three array with 5 cm spacing. The arrangement of the inserts is also shown in FIG. 6b. The materials used were tissue equivalent materials, including lung (LN10) 202, a hollow closed cylinder of cortical bone (SBD), with air in the centre 204, cortical bone (SB5) 206, rib bone (RB2) 208, internal bone (IB7) 210, adipose tissue (AP7) 212 and water equivalent plastic (WT1, WTe) 214a, b. These materials were used such that the exact compositions were known.

In the experimental set-up, the proton beam 2 was generated by an IBA cyclotron-based system (Proteus Plus), which provides proton beam scanning technique up to a maximum energy of 230 MeV. Energies down to 98 MeV may be obtained using a set of degraders at the exit of the accelerator, and energies down to 30 MeV may be obtained using a range shifter placed at the end of the snout, which is in the vicinity of the object 100.

For the experimental results in FIG. 6a, a proton beam 2 of 180 MeV was used, and the phantom 216 was placed on a rotating platform. The phantom 216 was centred at the beam isocentre and no range shifters were used.

The proton beam was pulsed to provide twenty one spots, each of 0.05 MU. The resulting beam profile of each spot was measured by a detector 3. The detector used for the experimental measurements was a flat panel detector (PerkinElmer, XRD 1620 xN CS) with a Single substrate amorphous silicon active TFT/diode array (PerkinElmer Technologies GmbH & Co. KG).

The spatial profile of the beam measured for each spot was then processed as discussed above, to generate the α, β, γ and Dₜₒₜ parameters, as discussed above.

After each measurement, the phantom 216 was rotated 10 degrees and the process repeated, so that the entire phantom was scanned by the beam 2. This rotation also provided a range of different paths, where the path may intersect one or more of the inserts at a time.

The experimental set up was also replicated by a simulation, to provide the associated WEPL values. The measurements of the system provided the parameters (α, β, γ and Dₜₒₜ) associated with these WEPL. The combination of the measured parameters and simulated WEPLs was then used to train the neural network.

For the purposes of the simulation, the beam line was modelled using Fiorini, F., Schreuder, N. and Van den Heuvel, F., Technical Note: Defining cyclotron-based clinical scanning proton machines in a FLUKA Monte Carlo system. Med. Phys., 45 (2018), the contents of which is hereby incorporated by reference.

The phantom 216 including all its inserts was accurately simulated (composition and geometry recreated). One simulation per spot and per phantom rotation was carried out and the particle fluence was recorded for each phantom voxel (1×1×1 mm³). The proton stopping power was also recorded for each phantom material. Externally to the simulation process, each phantom voxel was converted into water equivalent thickness by using the stopping power information gathered from the simulations.

The final WEPL transversed by each beamlet was calculated by weighting the phantom WEPL with the particle fluence recorded in the related simulation. FIG. 6c is a representation of the simulation, with the detector 2 at the top of the Figure. FIG. 6c clearly shows one of the fan beamlets passing through the phantom 216.

FIG. 6a shows only one of the parameters, γ, against the WEPL provided in the simulation. Each point in FIG. 6a represents a single spot from the measurement process (and hence a single simulation). The process discussed above relates the WEPLs to the experimental parameters (alpha, beta, gamma and dose).

It will be appreciated that the phantom 216 discussed above is by way of example only. Any suitable arrangement of a phantom could be used for system calibration.

FIG. 5 illustrates a measurement apparatus in accordance with an alternative embodiment of the invention. The apparatus is the same as the apparatus discussed in relation to FIG. 1, unless stated otherwise.

The apparatus shown in FIG. 5 includes a second detector 5 that is provided between the proton beam source 1, and the object 100. The second detector 5 is a transparent proton beam detector that transmits the pencil beam 2 whilst also measuring it. Thus, once the beam 2 has passed through the second detector 5, it continues on to the object 100. The second detector 5 defines a second detector plane.

The second detector 5 may be any suitable transparent proton detector such as a Beam Gas Ionisation Profile Monitor developed for CERN.

Similar to the proton beam detector 1 discussed above, the second proton beam detector 5 measures the spatial distribution of the beam 2, and the energy deposited by the beam 2 as it passes through the detector 5.

The processor 4 fits the profile of the proton beam measured by the second proton beam detector 5 to a stable distribution. The stable distribution is described by the same characteristic function discussed above, and so the processor 4 is able to determine the parameters α₂, β₂ and γ₂ together with D_{tot, 2} based on the measurements made by the second detector 5. These parameters represent the beam 2 prior to it passing through the object 100.

As discussed above, the processor 4 also determines the parameters α₁, β₁ and γ₁ together with D_{tot, 1} for the beam 2 after it has passed through the object 100. The same distribution is used for the measurements made by both detectors 3, 5.

The processor 4 determines the difference between the parameters before the beam 2 has passed through the object 100, and after the beam 2 has passed through the object 100 to provide difference parameters α₂₋₁, β₂₋₁ and γ₂₋₁ together with D_{tot, 2-1}. The difference parameters can be used to determine the WEPL in the same manner as discussed above, where the difference parameters are used instead of the parameters determined from the measurements after the beam 2 has passed through the object 100.

The proton source 1 and proton detectors 3, 5 discussed above are given by way of example only. It will be appreciated that any suitable proton beam source 1 may be used to generate the pencil beam 2, and any suitable proton detectors 3, 5 may be used to measure the beam 2. The detector 3 provided after the object 100 may be a transmission detector or not.

The proton source can be one used for generating therapeutic proton beams; as such, it can be the same source that is used to treat a patient, and therefore only one source of both therapeutic and imaging energy may be needed. The processor 4 may be part of the control systems for the proton source 1 and so may be provided in the same housing, or may be provided as a separate computer (e.g. a desktop or laptop computer running a common operating system such as Microsoft Windows, Linux or Apple Mac OS X) or a server, either locally or remotely (“in the cloud”).

1. A measurement apparatus for determining a water equivalent path length (WEPL) through an object, the measurement apparatus comprising:
a proton beam source arranged to produce, in use, a beam of protons having a beam shape;
a proton detector, the proton detector defining a proton detection plane, the proton detector being arranged to measure:
a spatial profile of protons incident the proton detection plane; and
energy deposited inside the detector by protons incident on the proton detection plane
the proton detector further arranged to provide a signal indicative of the measured energy with the spatial profile; and
a processor coupled to the proton detector so as to process the signal;
in which the proton beam source and the proton detector define between them a space for the object, and in which the processor is arranged to process the signal so as to fit the spatial profile and deposited energy measured after the proton beam has passed through the object to a distribution having parameters, and from the parameters estimate a water equivalent path length of the object.

2. The apparatus of claim 1, in which the distribution is a stable distribution.

3. The apparatus of claim 2, in which the stable distribution is defined by its characteristic function, given by:
φ(t; α, β, γ, δ)=exp[itδ−|γt|^{α}(1−iβ sgn(t)ϕ)]
with ϕ(t)=tan(πα/2) except for α=1, where
$$ϕ = - \frac{2}{π} log ( t )$$
where the parameters comprise:
a first parameter, α,
a second parameter, β, and
a third parameter, γ;
and δ represents the position of the beam on the proton detector and sgn(t) is the sign function.

4. The apparatus of claim 3, in which
the first parameter has a value between 0 and 2 inclusive and is indicative of the shape of the beam as incident on the proton detector;
the second parameter has a value between −1 and 1 inclusive and is a measure of the symmetry of the beam as incident on the proton detector; and
the third parameter has a value greater than or equal to 0 and less than positive infinity represents the broadness of the distribution.

5. The apparatus of claim 4, in which the processor is arranged to determine an integrated proton dose deposited in the proton detector by protons incident on the proton detector for the beam, and to use the integrated proton dose to determine the WEPL.

6. The apparatus of claim 5, in which the processor is arranged to determine the WEPL by using the first, second and third parameters and the integrated proton dose as the inputs to a trained neural network.

7. The apparatus of claim 1, in which the proton beam source is a therapeutic proton beam source.

8. The apparatus of claim 1, further including a second proton detector, provided between the proton beam source and the object, the second proton detector being arranged to measure:
a spatial profile of protons incident on a proton detection plane of the second proton detector; and
energy deposited inside the second proton detector by protons incident on the second proton detection plane
the second proton detector further arranged to provide a signal indicative of the measured energy with the spatial profile
in which the processor is further arranged to: fit the spatial profile and deposited energy measured by the second proton detector before the proton beam has passed through the object to the distribution, and based on a difference between the parameters determined before the proton beam passes through the object, and after the proton beam passes through the object, estimate a water equivalent path length of the object.

9. A method of determining a water equivalent path length (WEPL) through an object, the method comprising:
passing a beam of protons having a beam shape through the object; and
detecting, with a proton detector defining a proton detection plane:
a spatial profile of protons incident on the proton detection plane; and
energy deposited inside the proton detector by protons incident on the proton detection plane;
in which the proton beam is incident on the proton detection plane after it has passed through the object
the method further comprising fitting the detected spatial profile and deposited energy to a distribution having parameters and from the parameters estimating a water equivalent path length of the object.

10. The method of claim 9, in which the distribution is a stable distribution.

11. The method of claim 10, in which the stable distribution is defined by its characteristic function, given by:
φ(t; α, β, γ, δ)=exp[itδ−|γt|^{α}(1−iβ sgn(t)ϕ)]
where the parameters comprise:
a first parameter, α,
a second parameter, β, and
a third parameter, γ;
and δ represents the position of the beam on the proton detector and sgn(t) is the sign function.

12. The method of claim 11, in which:
the first parameter has a value between 0 and 2 inclusive and is indicative of the shape of the beam as incident on the proton detector;
the second parameter has a value between −1 and 1 inclusive and is a measure of the symmetry of the beam as incident on the proton detector; and
the third parameter has a value greater than or equal to 0 and less than positive infinity represents the broadness of the distribution.

13. The method of claim 12, comprising determining an integrated proton dose deposited in the detector by protons incident on the proton detection plane for the beam, and using the integrated proton dose to determine the WEPL.

14. The method of claim 13, comprising determining the WEPL by using the first, second and third parameters and the integrated proton dose as the inputs to a trained neural network.

15. The method of claim 9, including:
prior to passing the beam of protons through the object, detecting:
a spatial profile of protons incident on a second proton detection plane; and
energy deposited inside a second proton detector by protons incident on the second proton detection plane;
the method further comprising fitting the detected spatial profile and deposited energy to the distribution and from the difference between the parameters detected before and after the beam passes through the object estimating a water equivalent path length of the object.
